(19) United States
(12) Patent Application Publication
(10) Pub. No.: US 2023/0389970 A1
(43) Pub. Date: Dec. 7, 2023

(54) Bone Screw Apparatus and Method

(71) Applicant: SeaSpine Orthopedics Corporation, Carlsbad, CA (US)

(72) Inventors: Corey Noel Gladieux, Carlsbad, CA (US); Shaeffer Bannigan, Carlsbad, CA (US)

(21) Appl. No.: 18/327,161

(22) Filed: Jun. 1, 2023

Related U.S. Application Data
(60) Provisional application No. 63/348,545, filed on Jun. 3, 2022.

(51) Int. Cl. A61B 17/86

(52) U.S. Cl. CPC A61B 17/864; A61B 17/8625

(57) ABSTRACT

A bone screw apparatus and method. The bone screw may include one or more bone collecting channels. The channels may include one or more cutting edges/surfaces. The bone screw channels may capture a variety of displaced bone materials. The bone screw may have a reduced insertion torque. The one or more bone collecting channels may have a curved bottom.

### CROSS-REFERENCE TO RELATED APPLICATIONS

This application claims the benefit of U.S. Provisional Application No. 63/348,545, filed Jun. 3, 2022. The entire contents of the aforementioned application are herein incorporated by reference in their entirety.

### TECHNICAL FIELD

The present embodiments relate generally to bone screws.

### BACKGROUND

Typical bone/orthopedic screws (e.g. pedicle screw) may compress and/or damage surrounding bone material when inserting into a target bone section, thereby increasing the torque required to insert the bone screw. Thus, there is a need to reduce the compression of bone material, reduce bone damage to surrounding bone material, and/or reduce the insertion torque of the bone screw.

The present invention is directed at overcoming, or at least improving upon, the disadvantages of the prior art.

### SUMMARY

In some embodiments of the invention, for example, a bone screw may include a screw shaft having one or more threads and one or more bone collecting channels. In some embodiments, the bone screw may include a screw head. In various embodiments, one or more bone collecting channels may include one or more cutting edges. In some embodiments, one or more bone collecting channels may have a depth from a crest of one or more threads to a radial distance less than a minor diameter of one or more threads. In various embodiments, one or more bone collecting channels may extend in a helical path. In some embodiments, one or more bone collecting channels may extend in a straight path. In various embodiments, one or more bone collecting channels may extend from a distal end of the bone screw towards a head of the bone screw. In some embodiments, one or more bone collecting channels may extend along a tapered end of the bone screw. In various embodiments, one or more bone collecting channels may capture bone material within a volume of one or more bone collecting channels. In addition, in some embodiments, the bone screw may be a pedicle screw. In various embodiments, one or more bone collecting channels may extend through two adjacent threads. In some embodiments, one or more bone collecting channels may extend through a root between the two adjacent threads. In various embodiments, one or more bone collecting channels may be two bone collecting channels annularly spaced about an axis of the bone screw. Further, in some embodiments, one or more bone screws may be in combination with a medical system. In various embodiments, a bottom wall of one or more bone collecting channels may be arcuate in shape. In some embodiments, one or more bone collecting channels may extend for about half of a length of the shaft.

In various embodiments, the bone screw may include one or more implant-grade materials. In some embodiments, one or more implant-grade materials may be, but is not limited to, carbon fiber, stainless steel, titanium, titanium alloys, aluminum, aluminum alloys, cobalt chromium, molybdenum, molybdenum alloys, nickel, nickel alloys, silicone, silicone blends, fluoropolymer, plastics, polyetheretherketone (PEEK), polyphenylene sulfide, polyphenylsulfone, fluorinated ethylene propylene, polychlorotrifluorylethylene, perfluoroalkoxy, ethylene tetrafluoroethylene, ethylene chlorotrifluoroethylene, and/or combinations thereof. In some embodiments, the bone screw may have an insertion torque in the range of about 1.5 in-lbs to about 12 in-lbs. In various embodiments, the insertion torque may be 8.2 in-lbs.

In some embodiments of the invention, for example, a cannulated bone screw may include a screw shaft having one or more threads and one or more bone collecting channels, wherein the one or more bone collecting channels are separated from a central bore of the cannulated bone screw. In some embodiments, the cannulated bone screw may include a screw head. In various embodiments, one or more bone collecting channels may include one or more cutting edges. In some embodiments, one or more bone collecting channels of a cannulated bone screw may have a depth from a crest of one or more threads to a radial distance less than a minor diameter of one or more threads. In various embodiments, one or more bone collecting channels of a cannulated bone screw may extend in a helical path. In some embodiments, one or more bone collecting channels of a cannulated bone screw may extend in a straight path. In various embodiments, one or more bone collecting channels of a cannulated bone screw may extend from a distal end of the cannulated bone screw towards a head of the cannulated bone screw. In some embodiments, one or more bone collecting channels of a cannulated bone screw may extend along a tapered end of the cannulated bone screw. In various embodiments, one or more bone collecting channels of a cannulated bone screw may capture bone material within a volume of one or more bone collecting channels. In addition, in some embodiments, the cannulated bone screw may be a cannulated pedicle screw. In various embodiments, one or more bone collecting channels of a cannulated bone screw may extend through two adjacent threads. In some embodiments, one or more bone collecting channels of a cannulated bone screw may extend through a root between the two adjacent threads. In various embodiments, one or more bone collecting channels of a cannulated bone screw may be two bone collecting channels annularly spaced about an axis of the cannulated bone screw. Further, in some embodiments, one or more canulated bone screws may be in combination with a medical system. In various embodiments, a bottom wall of one or more bone collecting channels of a cannulated bone screw may be arcuate in shape. In some embodiments, one or more bone collecting channels of a cannulated bone screw may extend for about half of a length of the shaft.

In various embodiments, the cannulated bone screw may include one or more implant-grade materials. In some embodiments, one or more implant-grade materials of a cannulated bone screw may be, but is not limited to, carbon fiber, stainless steel, titanium, titanium alloys, aluminum, aluminum alloys, cobalt chromium, molybdenum, molybdenum alloys, nickel, nickel alloys, silicone, silicone blends, fluoropolymer, plastics, polyetheretherketone (PEEK), polyphenylene sulfide, polyphenylsulfone, fluorinated ethylene propylene, polychlorotrifluorylethylene, perfluoroalkoxy, ethylene tetrafluoroethylene, ethylene chlorotrifluoroethylene, and/or combinations thereof. In some embodiments, the canulated bone screw may have an insertion torque in the range of about 1.5 in-lbs to about 12 in-lbs. In various embodiments, the insertion torque of a cannulated bone screw may be 8.2 in-lbs.

In some embodiments, a method of inserting a bone screw may include providing one or more bone screws having one or more bone collecting channels. In various embodiments, the method may include inserting one or more bone screws into a bone.

In addition, in various embodiments, the method may include reducing an insertion torque. In some embodiments, the method may include cutting bone material with one or more bone collecting channels. In various embodiments, the method may include cutting bone material for over about half a length of a shaft and/or threads. In some embodiments, the method may include collecting bone material into one or more bone collecting channels. In various embodiments, the method may include steering one or more bone screws when inserting. In some embodiments, the method may include drilling a pilot hole for one or more bone screws when inserting. In various embodiments, the method may not include providing a pilot hole for one or more bone screws when inserting. In some embodiments, the method may include self-tapping a distal end of the screw.

In some embodiments, a method of inserting a cannulated bone screw may include providing one or more cannulated bone screws having one or more bone collecting channels. In various embodiments, the method may include inserting one or more cannulated bone screws into a bone.

In addition, in various embodiments, the method may include reducing an insertion torque of a cannulated bone screw. In some embodiments, the method may include cutting bone material with one or more bone collecting channels of a cannulated bone screw. In various embodiments, the method may include cutting bone material for over about half a length of a shaft and/or threads of a cannulated bone screw. In some embodiments, the method may include collecting bone material into one or more bone collecting channels of a cannulated bone screw. In various embodiments, the method may include steering one or more cannulated bone screws when inserting. In some embodiments, the method may include drilling a pilot hole for one or more cannulated bone screws when inserting. In various embodiments, the method may not include providing a pilot hole for one or more cannulated bone screws when inserting. In some embodiments, the method may include self-tapping a distal end of the cannulated screw.

In some embodiments, a kit may include one or more bone screws having one or more bone collecting channels. In various embodiments, the kit may include one or more insertion devices. In some embodiments, the kit may include a set of instructions.

In some embodiments, a kit may include one or more cannulated bone screws having one or more bone collecting channels. In various embodiments, the kit including a cannulated bone screw may include one or more insertion devices. In some embodiments, the kit including a cannulated bone screw may include a set of instructions.

### BRIEF DESCRIPTION OF THE ILLUSTRATIONS

In the drawings, like reference characters generally refer to the same parts throughout the different views. Also, the drawings are not necessarily to scale, emphasis instead generally being placed upon illustrating the principles of the invention.

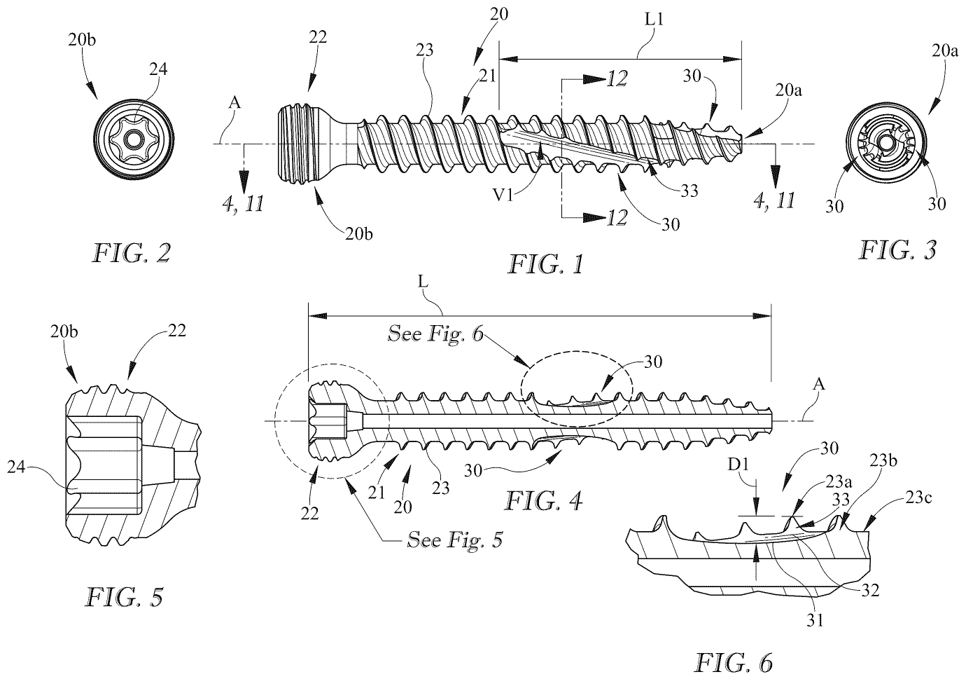

FIG. 1 is a side view of one embodiment of a bone screw.

FIG. 2 is an end view of the proximal end of the bone screw of FIG. 1.

FIG. 3 is an end view of the distal end of the bone screw of FIG. 1.

FIG. 4 is a side sectional view of the bone screw of FIG. 1 taken along line 4-4.

FIG. 5 is an enlarged sectional view of FIG. 4.

FIG. 6 is an enlarged sectional view of FIG. 4.

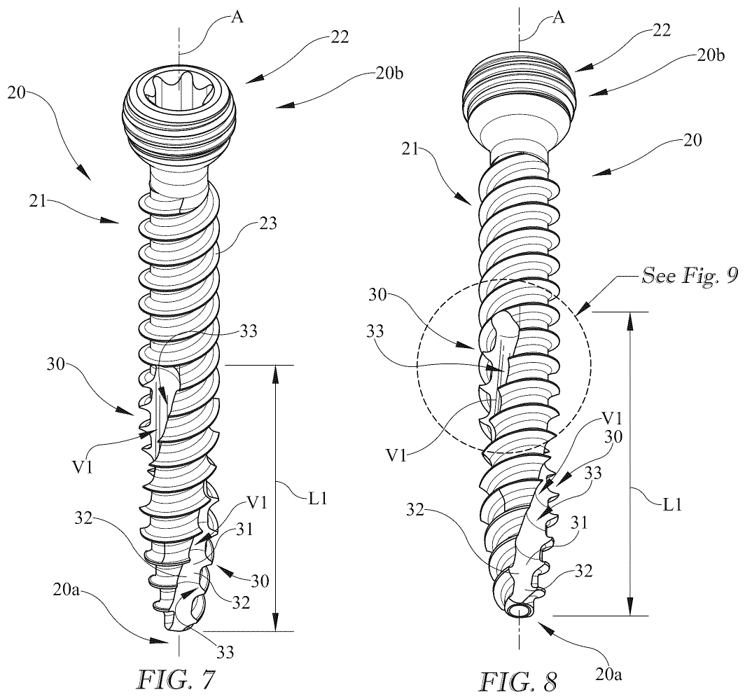

FIG. 7 is a top perspective view of the bone screw of FIG. 1.

FIG. 8 is a bottom perspective view of the bone screw of FIG. 1.

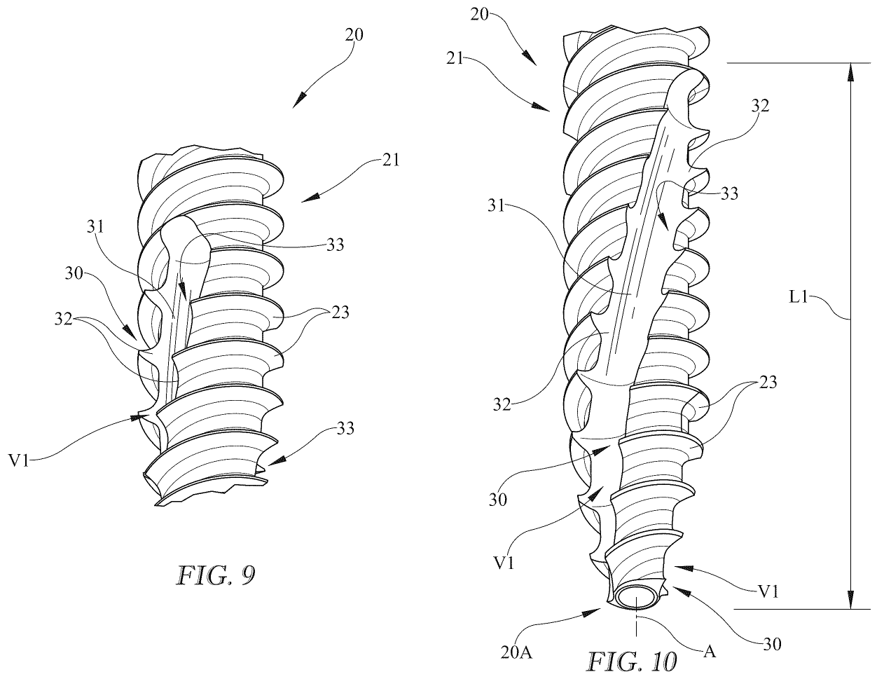

FIG. 9 is an enlarged perspective view of the bone collecting channel of FIG. 8.

FIG. 10 is another enlarged perspective view of the bone collecting channel.

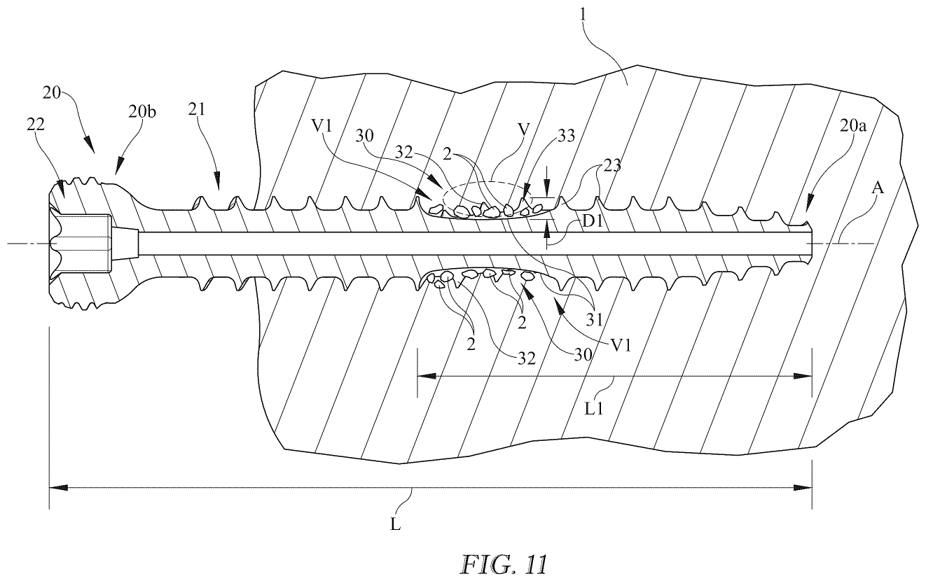

FIG. 11 is a side sectional view of the bone screw of FIG. 1 taken along line 11-11 inserted into a bone, illustrating the capture of bone material cut, displaced, or removed from the bone.

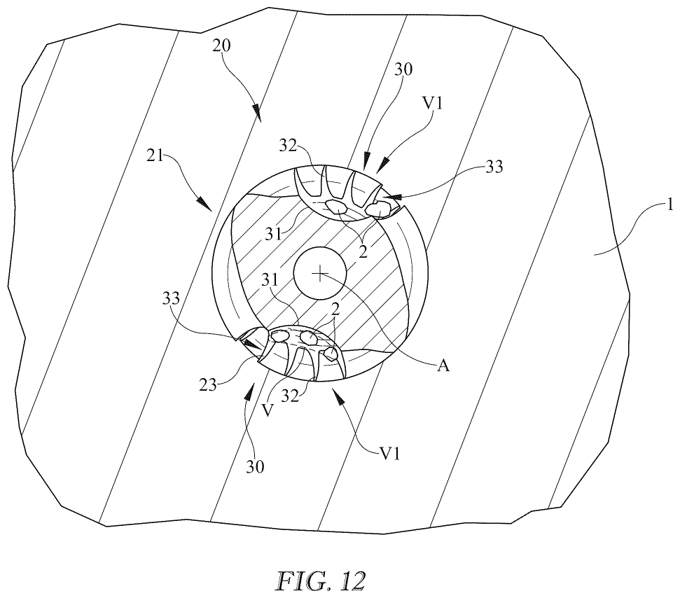

FIG. 12 is a sectional view of the bone screw of FIG. 1 taken along line 12-12 inserted into a bone, illustrating the capture of bone material cut, displaced, or removed from the bone.

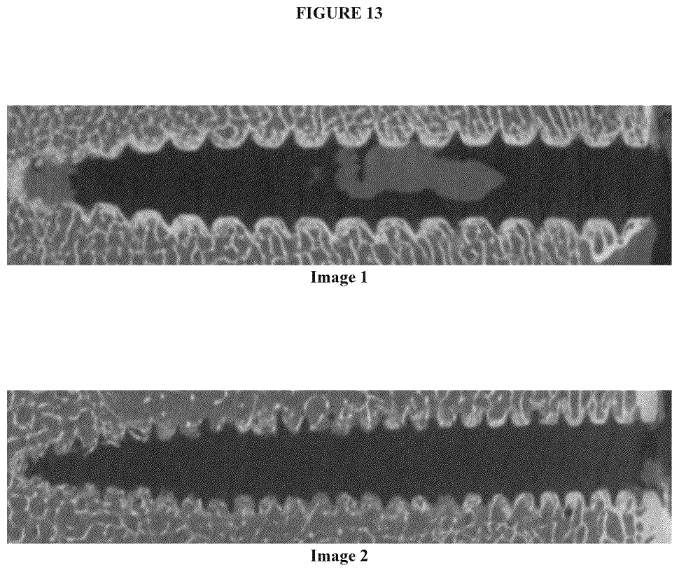

FIG. 13 provides a pair of comparative images (Image 1 and Image 2) illustrating results from a study conducted on bovine condyles.

### DETAILED DESCRIPTION

Embodiments may further be understood with reference to the various Figures. With reference to Figures, an embodiment provides a bone screw 20 that may possess a shaft 21, and a screw head 22 that may be integral with or attached to the shaft 21. The screw head 22 may be a portion of a sphere or have a spheroidal shape. The apparatus may further be provided with a collet (not shown) that may fit around all or a portion of the screw head 22. The apparatus may further have a movable head (not shown), which may in turn fit around the collet. Although a pedicle screw is shown in the one embodiment, it should be understood that a variety of bone screws may be used and still be within the scope of the invention. For example, the bone screw may be a lag screw. The bone screw may be cannulated or uncannulated. Further, for example, the bone screw may be an intervertebral screw for an intervertebral body/cage/implant. The bone screw may be used in a variety of applications/bones such as, but is not limited to, in combination with medical devices or systems (e.g. implants, plates, cages, etc.), vertebral bodies, femur, arm bones, hand, feet, wrist, ankles, leg bones, pelvic, etc. The one or more bone screws may be inserted by one or more insertion devices. Further, a set of instructions (e.g. how to insert bone screw and/or other devices, installation, etc.) may be used or combined with the one or more bone screws, kit, insertion devices, and/or medical devices, or portions thereof.

The bone screw may be of a variety of shapes, profiles, sizes, lengths, diameters, constructions, and materials. The one or more materials may be any implant-grade material. For example, carbon fiber reinforced PEEK (CFRP) may be used, as well as, other PEEK and/or alloys. Further, a titanium alloy may be used. It should be understood, additional materials not typically used in bone screws may be used because the reduction of insertion torque affords the use of implant-grade materials that would otherwise be unsuitable due to inability to withstand significant torque loading. For example, biocompatible materials may be used in some applications. In one or more embodiments, materials for constructing bone screws according to the present disclosure includes 3D-printed screws.

In some implementations, components of the presently described bone screw and/or insertion device may be manufactured of various materials, including clinical grade materials. Example materials include, but are not limited to, carbon fiber, various metals and alloys thereof, including stainless steel, titanium, titanium alloys, aluminum, aluminum alloys, cobalt chromium, molybdenum, molybdenum alloys, nickel, nickel alloys, and/or combinations thereof. Silicone and silicone blends may also be used to fabricate one or more component(s) of the bone screw and/or insertion device. Further, materials of construction for one or more of the components of the bone screw and/or insertion device include fluoropolymer and other plastics. Examples include PEEK (polyetheretherketone), PPS (polyphenylene sulfide), PPSU (polyphenylsulfone), FEP (fluorinated ethylene propylene), PCTFE (polychlorotrifluorylethylene), PFA (perfluoroalkoxy), ETFE (ethylene tetrafluoroethylene), ECTFE (ethylene chlorotrifluoroethylene), and the like. Composites and/or combinations thereof of the above or similar materials may also be employed.

In particular, materials of construction employed in one or more components of the presently described bone screw and/or insertion device are able to withstand autoclaving, including parameters such as saturated steam under pressure, (˜1 atm), along with concomitant autoclave chamber temperatures ranging from about 100° C. to 150° C. for about 15 to 60 minutes. Other relevant autoclaving and/or sterilization procedures and temperatures may also be employed.

Referring now to the Figures in additional detail, screw 20 may possess threads 23 around shaft 21. Screw head 22 may also have, at its end opposite shaft 21, a tool interface recess 24 (see, e.g., FIG. 2) that may be a hexalobe feature. Screw 20 may have a longitudinal axis A. The longitudinal axis A generally extends through the center of the screw 20 along its length. In the vicinity of longitudinal axis A, the screw shaft 21 may be either solid or alternatively may be hollow (as illustrated), with the empty central region being available for other purposes as may be desired.

In some implementations, the bone screw 20 may include one or more bone collecting reservoirs, troughs, channels, cut-aways, and/or voids 30. The bone collecting channel may cut and/or collect bone chips or material 2 removed from the screw path when inserting into the bone 1 (e.g. material). The outward bone displacement or bone material compression is reduced because the bone collecting channel 30 receives fragmented bone material 2 (e.g. dust, chip, debris). The decrease of the bone compression may result in reducing the friction when inserting the bone screw 20, and correspondingly reduce the amount of torque force for inserting the bone screw in the bone 1. The amount of torque force for insertion into the target bone section may be in the range of about 1.5 in-lbs to about 12 in-lbs. One example of the peak insertion torque may be, but is not limited to, about 8.2 in-lbs. This may be a reduction of torque within the range of about 75 to about percent as compared to a typical pedicle screw in some embodiments.

In some implementations, the one or more bone collecting channels may extend along one or more lengths L1 along the length L or axis A of the screw 20, shaft 21, or threads 23. The channel 30 may extend from the distal end 20a, opposite the head 22, for one or more lengths L1. The channel 30, or portions thereof, (e.g. one end) may be positioned adjacent the screw tip or distal end 20a to assist in self-starting or self-tapping the screw into the bone material 2. Although the tip or distal end is tapered in the one embodiment, a variety of tips (e.g. shapes, sizes, etc.) may be used and still be within the scope of the invention. The channel 30 may extend along the tapered or distal end of the bone screw as shown in the one embodiment. As shown in the one embodiment, the channel 30 does not have to extend from the distal end 20a to the head 22. The opposing end of the channel 30 may be spaced from the head or proximal end 20b of the screw. The length L1 of the channel 30 may extend about half the length of the threads 23 and/or the shaft/screw in some embodiments. Although not shown, the channel 30 or opposing end of the channel may extend for a length that is spaced from the distal end 20a or not positioned on the distal end 20a. Although one or more single channels extend for a length L1, it should be understood that a plurality of channels, having the same or different lengths, may be combined to form a path or discontinuous channel (e.g. helical, straight) along the axis A. Although two channels 30 are shown in the embodiment annularly spaced around the axis A and extend for a length L1 along the length L of the screw 20, threads 23, or shaft 21, it should be understood that one or more channels 30 may be used in some embodiments. Two or more channels, on the same screw body or shaft, may be the same or different. For example, the length, depth, shape, orientation, and/or position may be different or the same between two or more channels.

The one or more channels may include one or more paths along the length or axis A of the screw. As shown in the one embodiment, the channel paths are helical in shape. The helical path or channel cuts/extends/passes through the helical threads along the axis A. The channel 30 may create one or more discontinuous threads along the axis A. The helical path of the channel may be in the same or similar direction as the thread as shown in the one embodiment. It should be understood that a variety of paths (e.g. for one or more lengths L1) may be used. For example, the paths may be linear or straight (e.g. nonparallel or parallel to the axis) in some embodiments along the longitudinal axis A.

In some implementations, the one or more bone collecting channels 30 may define or include a volume V1 (e.g. cutaway) to collect the bone material 2 that is removed/cut/displaced. The channel 30, or cross section thereof, may include a depth D1 and/or size that is cut or removed from the screw, or portions thereof, (e.g. threads, body, shaft). Although the shape of the channel 30 or bottom wall 31 of the channel is arcuate, it should be understood that the bottom and/or sidewalls 32, connecting to the bottom wall 31, may be a variety of shapes, depths, sizes, and quantities to collect/cut/separate bone pieces. For example, the bottom wall or cross section may be rectangular in some embodiments. Further, the opposing sidewalls may be the same as shown in the Figures or different. For example, the trailing sidewall or cutting edge 33 may be different from the leading edge or sidewall in some embodiments. The one or more depths D1 along the one or more lengths L1 may be varied or constant. One or more of the opposing ends of the channel may be tapered. It should be understood that the shape, length, depth, quantity, or other characteristics of the channel may be varied to correspondingly cut/contain/capture a variety of volumes V of displaced/cut bone pieces/fragments 2 in one or more applications. The depth D1 of the channel may extend from or through the crest 23a (e.g. outer most periphery at the threads or major diameter) of the thread towards the axis A of the screw. As shown in the one embodiment, the depth D1 of the channel 30 may be greater than the depth of the thread (e.g. distance between the crest 23a and base 23b of the thread) and/or extend/project (e.g. radially inward) within/past/into the minor diameter of the thread/screw and/or root as shown in FIG. 6. The depth D1 of the channel 30 may extend to a radial distance less than the minor diameter of the thread. In some embodiments, the depth D1 of the channel 30 along a length L1, or portions thereof, may be less than the depth of the thread and extend through opposing sides/flanks of the thread. For example, at the one or more opposing ends of the channel. The one or more channels at one or more depths D1 may cut/pass/extend through one or more threads (e.g. adjacent, non-adjacent) and/or one or more surfaces of the roots of the screw along the axis A.

In one or more embodiments, bone screw 20 comprises a cannulated screw. One or more bone collecting channel(s) 30 may be segregated from the central bore of such a cannulated bone screw 20. For example, FIG. 10 illustrates the distal end portion of a central bore 20A of a cannulated bone screw 20. In this embodiment, depth D1 of channel 30 does not penetrate into the central bore 20A of cannulated bone screw 20. Further, bottom wall 31 in this embodiment functions to block bone material 2 from entering central bore 20A. Also, FIG. 11 illustrates a cross-sectional view of bone screw 20 in which bone material 2 that has been removed/cut/displaced resides in channel 30. Although the shape of channel 30 or bottom wall 31 of the channel may be arcuate, whether in a solid or cannulated bone screw, it should be understood that the bottom and/or sidewalls 32, connecting to the bottom wall 31, may be a variety of shapes, depths, sizes, and quantities to collect/cut/separate bone pieces. For example, the bottom wall or cross section may be rectangular in one or more embodiments. Further, the opposing sidewalls may be the same as shown in the Figures or different. For example, the trailing sidewall or cutting edge 33 may be different from the leading edge or sidewall in some embodiments.

In some implementations, the screw 20 and/or channel 30 may include one or more cutting edges/surfaces 33. The cutting edge/surface 33 may cut or remove bone 2 from the path (e.g. insertion) of the screw 20. The cut/removed/separated bone 2 from the cutting edge 33 or channel 30 rotation may be a variety of sizes and/or shapes. The cut/removed/separated bone 2 or volume V may be collected within the one or more channels 30 (e.g. volume V1). The volume V1 of the one or more channels 30 may be filled at least partially by the bone collected 2 (e.g. volume V). The cutting edge/surface may be circumferential, serrated, or straight path cutting. The one or more cutting edges 33 may be positions on the trailing edge or surface of the one or more channels 30 in the direction of rotation (e.g. clockwise) of the screw during insertion. The cutting edges/surfaces (e.g. sidewall 32, channel 30, or portions thereof, crest 23a, base 23b, and/or root 23c) may extend from the outer diameter or major diameter of the channel to the inner diameter or bottom wall 31 of the channel. The cutting edge/surfaces may extend from the crest of the thread to the bottom of the channel (e.g. radially inward from the root 23c or minor diameter). The cutting edge 33 may include the channel 30, or portions thereof, (e.g. the crest 23a, one or more sidewalls 32 of the thread, and/or the root 23c between the threads, and/or threads). The cutting edge/surface may be continuous or discontinuous along the length L of the screw.

In use in some applications, the bone screw may not require a pilot hole before insertion. The bone screw may have easier insertion that may reduce the need for a pilot hole. Alternatively, in some embodiments, a pilot hole of various depths into the target bone may be used or drilled into the bone 1 before insertion of the bone screw 20. The one or more channels may be used with screws having a variety of pitches, screw profiles, one or more thread starts, diameters, lengths, sizes, etc. for one or more applications. In some embodiments, the one or more channels with collected/cut bone material may promote bone growth into the volume V and/or receive graft material.

FIG. 13 illustrates that the bone preparation techniques and implant screw design have a major influence on boney necrosis via cellular compression, affecting how the bone/implant interface heals, and potentially leading to screw loosening. This experiment involved preparing bovine condyles with various bone preparation techniques and screw designs. The bone was prepared according to the technique, screws were inserted and removed, and then Micro CT images of the bone were generated to assess the compression.

Again referring to FIG. 13, traditional pedicle screw preparation techniques include creating a pilot hole with an awl, expanding the pilot hole with a probe, tapping 1 mm undersized the desired screw implant size, and then inserting a traditional screw implant designed without the subject bone displacing flute technology. In this experiment, the method of pedicle screw preparation included creating a pilot hole with an awl and inserting the novel pedicle screw of this disclosure which includes the subject bone displacing flute technology.

In FIG. 13, Image 1 illustrates a Micro CT scan of traditional pedicle preparation and screw insertion techniques. Further, Image 2 illustrates a Micro CT scan of the presently described pedicle preparation technique in conjunction with the novel screw design as provided herein.

The FIG. 13 images are results from this study conducted on bovine condyles. In this study, a traditional bone preparation technique and screw design (Image 1) were compared against the presently described method and screw design (Image 2) as disclosed herein. The present method and screw design (Image 2) of this disclosure results in comparatively less compressed bone than the traditional bone preparation technique and screw design (Image 1) as shown by the decreased white area around the thread impressions.

The compression caused by the screw insertion changes the native architecture of the bone. This can result in a stronger bond of the implant/bone interface initially but may lead to screw loosening as the bone remodels back to the native architecture. The present method and screw design of this disclosure results in less compressed bone volume as compared to traditional preparation techniques and screw designs, resulting in a faster time to heal and thus a faster adhesion of the bone to the implant.

While several embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, embodiments may be practiced otherwise than as specifically described and claimed. Embodiments of the present disclosure are directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

It is to be understood that the embodiments are not limited in its application to the details of construction and the arrangement of components set forth in the description or illustrated in the drawings. The invention is capable of other embodiments and of being practiced or of being carried out in various ways. Unless limited otherwise, the terms “connected,” “coupled,” “in communication with,” and “mounted,” and variations thereof herein are used broadly and encompass direct and indirect connections, couplings, and mountings. In addition, the terms “connected” and “coupled” and variations thereof are not restricted to physical or mechanical connections or couplings.

The foregoing description of several embodiments of the invention has been presented for purposes of illustration. It is not intended to be exhaustive or to limit the invention to the precise steps and/or forms disclosed, and obviously many modifications and variations are possible in light of the above teaching.

1. A bone screw comprising:
a screw shaft having one or more threads and one or more bone collecting channels.

2. The bone screw of claim 1 wherein the one or more bone collecting channels includes one or more cutting edges.

3. The bone screw of claim 1 wherein the one or more bone collecting channels has a depth from a crest of the one or more threads to a radial distance less than a minor diameter of the one or more threads.

4. The bone screw of claim 1 wherein the one or more bone collecting channels extends in a helical path.

5. The bone screw of claim 1 wherein the one or more bone collecting channels extends in a straight path.

6. The bone screw of claim 1 wherein the one or more bone collecting channels extends from a distal end of the bone screw towards a head of the bone screw.

7.-17. (canceled)

18. The bone screw of claim 1 having an insertion torque in the range of about 1.5 in-lbs to about 12 in-lbs.

19. (canceled)

20. A cannulated bone screw comprising:
a screw shaft having one or more threads and one or more bone collecting channels;
wherein said one or more bone collecting channels are separated from a central bore of said cannulated bone screw.

21. The cannulated bone screw of claim 20 wherein the one or more bone collecting channels includes one or more cutting edges.

22. The cannulated bone screw of claim 20 wherein the one or more bone collecting channels has a depth from a crest of the one or more threads to a radial distance less than a minor diameter of the one or more threads.

23. The cannulated bone screw of claim 20 wherein the one or more bone collecting channels extends in a helical path.

24. The cannulated bone screw of claim 20 wherein the one or more bone collecting channels extends in a straight path.

25. The cannulated bone screw of claim 20 wherein the one or more bone collecting channels extends from a distal end of the bone screw towards a head of the bone screw.

26.-36. (canceled)

37. The cannulated bone screw of claim 20 having an insertion torque in the range of about 1.5 in-lbs to about 12 in-lbs.

38. (canceled)

39. A method of inserting a bone screw comprising the steps of:
providing one or more bone screws having one or more bone collecting channels; and
inserting the one or more bone screws into a bone.

40. (canceled)

41. The method of claim 39 further comprising the step of cutting bone material with the one or more bone collecting channels.

42. (canceled)

43. The method of claim 41 further comprising the step of collecting bone material into the one or more bone collecting channels.

44.-50. (canceled)

51. A method of inserting a cannulated bone screw comprising the steps of:
providing one or more cannulated bone screws having one or more bone collecting channels; and
inserting the one or more cannulated bone screws into a bone.

52. (canceled)

53. The method of claim 51 further comprising the step of cutting bone material with the one or more bone collecting channels.

54. (canceled)

55. The method of claim 53 further comprising the step of collecting bone material into the one or more bone collecting channels.

56.-62. (canceled)
